(19) United States
(12) Patent Application Publication
(10) Pub. No.: US 2009/0030407 A1
(43) Pub. Date: Jan. 29, 2009

(54) Apparatus and Method to Predict Individual Maximum Safe Radiant Exposure (IMSRE) Based on Measurement of Temporal Temperature Increase Induced by a Sub-Therapeutic Laser Pulse

(75) Inventors: Willem Verkruysee, Costa Mesa, CA (US); J. Stuart Nelson, Laguna Niguel, CA (US)

Correspondence Address: Law Offices of Daniel L. Dawes, 5200 Warner Blvd, Ste. 106, Huntington Beach, CA, 92649, US

(73) Assignee: THE REGENTS OF THE UNIVERSITY OF CALIFORNIA, Oakland, CA (US)

(21) Appl. No.: 12/178,548

(22) Filed: Jul. 23, 2008

Related U.S. Application Data
(60) Provisional application No. 60/951,586, filed on Jul. 24, 2007.

(51) Int. Cl. A61B 18/20

(52) U.S. Cl. 606/9; 606/12

(57) ABSTRACT

A method for making pulsed photothermal radiometric measurements to determine individual maximum safe radiant exposure (IMSRE) of biological subjects corresponding to radiant energy exposure (RE) without any use of a biological model includes a calibration procedure, including the steps of applying a statistical regression to an empirical data set of IMSRE and temporal REs applied to a sample population of the subjects to determine a IMSRE corresponding to each temporal RE. The IMSRE is set so that using the statistical regression separation of the data set into an acceptable injury grouping and an unacceptable injury grouping is obtained with a predetermined limitation of the proportion of subjects having unacceptable injury at a temporal RE below the corresponding IMSRE. The separation of the data set is thus used to predict an IMSRE for a corresponding temporal RE to a biological subject not included in the sample population.

### RELATED APPLICATIONS

The present application is related to U.S. Provisional Patent Application, Ser. No. 60/951586, filed on Jul. 24, 2007, which is incorporated herein by reference and to which priority is claimed pursuant to 35 USC 119.

### BACKGROUND OF THE INVENTION

1. Field of the Invention

The invention relates to the field of photodynamic use of radiation for treatment of tissue and in particular to the establishment of individual maximum safe radiant exposures in medical use of pulsed lasers.

2. Description of the Prior Art

With respect to safe levels of personal skin exposure to laser irradiation, the threshold question is how can an individual pulsed photothermal radiometric measurement be used to determine individual maximum safe radiant exposure. This question is illustrated schematically in FIG. 1, where five differently pigmented individuals are to the mapped to corresponding individual maximum safe radiant exposures (imsre). This question has been investigated, for example by Jung B J et.al. “Hand-held pulsed photothermal radiometry system to estimate epidermal temperature rise during laser therapy”, Skin Res. Technol. 2006;12:292-297 (Beckman Laser Institute group, UC Irvine). Jung states: . . .
. . . a maximum safe radiant exposure . . . can be defined above which epidermal thermal damage would occur. . . . A measure of epidermal heating would provide clinicians with an objective means to determine Hₘₐₓ. Pulsed photo-thermal radiometry (PPTR) can provide accurate measurements of epidermal heating.

Altshuler et al. U.S. Pat. No. 6,015,404 very generally describes a diagnostic feed-back to a laser system for use with systems applying laser energy to treat a selected dermatology problem. The method and apparatus protect skin not under treatment in skin regions affected by the laser by detecting, with a suitable sensor, at least a selected parameter in the skin region affected by the delivered laser energy and performing a control function to effect the desired protection by use of a feedback mechanism which is operative in response to an output from the sensor. For some embodiments, two laser pulses may be utilized, which pulses are spaced by a time which is preferably greater than the thermal relaxation time for affected regions not under treatment, for example an epidermis through which the energy is passed to an area under treatment, but is less than the thermal relaxation time of the area under treatment. The first of the pulses serves as a prediagnosis pulse which is clearly below the damage threshold for protected areas, with the sensor output for the first pulse being utilized to control at least one parameter of the second pulse.

So far, the published literature has only hinted at predicting the individual maximum safe radiant exposure, but have never disclosed an operable method or apparatus to actually make a reliable prediction. Previous publications have always aimed at quantifying the pigmentation and then implying that this number could then be used to determine the individual maximum safe radiant exposure, but without showing how. To implement the idea implicitly according to prior art approaches requires that a few modeling steps be involved:

invert the pulsed photo-thermal radiometric temporal signal to a depth profile of the chromophores (giving the melanin concentration in the epidermis); and

The result of step 1 is used in a forward model to calculate the threshold temperature at the basal layer (epidermal-dermal junction). If pre-cooling is involved, (which is common clinical practice) this process is required to be quantified as well, with a new array of uncertainties and assumptions.

Each of these steps involves determination, estimation or assumption of various skin parameters (optical, thermal and geometrical) and cryogen cooling parameters. The process also requires an assumption of a damage model. The resulting individual maximum safe radiant exposure depends critically on the accuracy of these assumptions. This is schematically illustrated in the FIG. 2 which illustrates the complexity of this approach.

The complexity of these two modeling steps has prevented researchers from actually using pulsed photo-thermal radiometric signals to predict individual maximum safe radiant exposure. Moreover, the above approach is very vulnerable for noise in the pulsed photo-thermal radiometric signal thereby reducing the robustness of the prediction.

### BRIEF SUMMARY OF THE INVENTION

The illustrated embodiment of the invention is an improvement in a method for making pulsed photothermal radiometric measurements to determine individual maximum safe radiant exposure (IMSRE) of biological subjects corresponding to radiant energy exposure (RE) without any use of a biological model. The method includes a calibration methodology, which comprises the steps of applying a statistical regression to an empirical data set of individual maximum safe radiant exposures (IMSRE) and temporal radiant energy exposures (RE) applied to a sample population of the subjects to determine a individual maximum safe radiant exposure (IMSRE) corresponding to each temporal radiant energy exposure (RE). The IMSRE is set so that using the statistical regression separation of the data set into an acceptable injury grouping and an unacceptable injury grouping is obtained with a predetermined limitation of the proportion of subjects having unacceptable injury at a temporal radiant energy exposure (RE) below the corresponding individual maximum safe radiant exposure (IMSRE). The separation of the data set is thus used to predict an individual maximum safe radiant exposure (IMSRE) for a corresponding temporal radiant energy exposure (RE) to a biological subject not included in the sample population.

The step of applying a statistical regression to an empirical data set of individual maximum safe radiant exposures (IMSRE) and temporal radiant energy exposures (RE) to determine a individual maximum safe radiant exposure (IMSRE) corresponding to each temporal radiant energy exposure (RE) comprises in one embodiment the step of applying a partial least squares (PLS) regression to quantify a relationship between the individual maximum safe radiant exposures (IMSRE) and temporal radiant energy exposures (RE) in the data set.

In another embodiment the step of applying a statistical regression to an empirical data set of individual maximum safe radiant exposures (IMSRE) and temporal radiant energy exposures (RE) to determine a individual maximum safe radiant exposure (IMSRE) corresponding to each temporal radiant energy exposure (RE) comprises the step of approximating the statistical regression by the relation:

$${IMSRE}_{i} = K \frac{{RE}_{D}}{Δ {T}_{i}} ,$$

where RE_{D} is a radiant exposure of a diagnostic laser pulse, which comprises the temporal radiant energy exposure (RE), where ΔTᵢ is a measured temperature increase at a predetermined time after the laser diagnostic pulse, and where K is an empirically determined calibration constant determined empirically on the basis of the data set.

The predetermined time after the laser diagnostic pulse comprises a time period at which contribution to heat absorption in the skin from the hair follicles is negligible while contribution to heat absorption in the skin from the melanin bearing epidermal layer is dominant over contribution to heat absorption in the skin from deeper chromophores.

In one embodiment the predetermined time after the single laser diagnostic pulse comprises a measurement at approximately 20 ms. In particular, the predetermined time after the single laser diagnostic pulse comprises a single measurement at approximately 20 ms.

In another embodiment the step of applying a statistical regression to an empirical data set of individual maximum safe radiant exposures (IMSRE) and temporal radiant energy exposures (RE) to determine a individual maximum safe radiant exposure (IMSRE) corresponding to each temporal radiant energy exposure (RE) comprises the step of approximating the statistical regression by an inverse proportionality relationship between individual maximum safe radiant exposures (IMSRE) and a temperature increase ΔT in targeted tissue in the subject induced by a sub-therapeutic laser pulse comprising the temporal radiant energy exposures (RE).

In one embodiment the step of applying a statistical regression to an empirical data set of individual maximum safe radiant exposures (IMSRE) and temporal radiant energy exposures (RE) to obtain a separation of the data set into an acceptable injury grouping and an unacceptable injury grouping with a predetermined limitation of the proportion of subjects having unacceptable injury at a temporal radiant energy exposure (RE) below the corresponding individual maximum safe radiant exposure (IMSRE) comprises the step of obtaining the separation of the data set with a limitation of 3% or less of the subjects having unacceptable injury at a temporal radiant energy exposure (RE) below the corresponding individual maximum safe radiant exposure (IMSRE).

In another embodiment the step of applying a statistical regression to an empirical data set of individual maximum safe radiant exposures (IMSRE) and temporal radiant energy exposures (RE) applied to a sample population of the subjects to determine a individual maximum safe radiant exposure (IMSRE) corresponding to each temporal radiant energy exposure (RE) comprises the step of applying a statistical regression to an empirical data set generated by employing a plurality of measurements over time starting from when a diagnostic laser pulse is applied to approximately one second thereafter to determine individual maximum safe radiant exposure (IMSRE).

The step of applying a statistical regression comprises the step of using partial least squares regression (PLS) to determine an individual maximum safe radiant exposure vector (IMSREᵢ) whose components are individual maximum safe radiant exposure values from the data set in which a predetermined damage threshold is just reached, where RE values that caused the predetermined damage threshold are used as the individual maximum safe radiant exposure values, and where Tᵢ is a vector whose components are reciprocal pulsed photo-thermal radiometric signals T_{I} corresponding to the individual maximum safe radiant exposure values in IMSRE_{I}, where K is a vector having the same length as Tᵢ and is determined using PLS from IMSREᵢ=K×Tᵢ, where IMSREᵢ is the matrix product K×Tᵢ.

The illustrated embodiment of the invention is also a method for applying a photothermal pulse to the skin of a patient with an individual maximum safe radiant exposure (IMSRE) without any use of a biological model. The photothermal pulse is applied to the skin with a radiant exposure at or below the individual maximum safe radiant exposure (IMSRE) as determined by using a statistical regression to an empirical data set of individual maximum safe radiant exposures (IMSRE) and temporal radiant energy exposures (RE) applied to a sample population of patients by obtaining a separation of the data set into an acceptable injury grouping and an unacceptable injury grouping with a predetermined limitation of the proportion of subjects having unacceptable injury at a temporal radiant energy exposure (RE) below the corresponding individual maximum safe radiant exposure (IMSRE), from which separation of the data set the individual maximum safe radiant exposure (IMSRE) for a corresponding temporal radiant energy exposure (RE) to the patient has been determined.

The illustrated embodiment of the invention is also an apparatus comprising a source of a photothermal pulse to be applied to the skin of a patient with an individual maximum safe radiant exposure (IMSRE) without any use of a biological model. A controller is coupled to the source where the radiant exposure provided by the photothermal pulse to the skin from the source as regulated by the controller is maintained at or below the individual maximum safe radiant exposure (IMSRE) as determined by using a statistical regression to an

In one embodiment the controller regulates the source to provide the photothermal pulse to the skin at or below the individual maximum safe radiant exposure (IMSRE) as determined by applying partial least squares (PLS) regression to quantify a relationship between the individual maximum safe radiant exposures (IMSRE) and temporal radiant energy exposures (RE) in the data set.

In another embodiment the controller regulates the source to provide the photothermal pulse to the skin at or below the individual maximum safe radiant exposure (IMSRE) as determined by approximating the statistical regression by the relation:

$${IMSRE}_{i} = K \frac{{RE}_{D}}{Δ {T}_{i}} ,$$

where RE_{D} is a radiant exposure of a diagnostic laser pulse, which comprises the temporal radiant energy exposure (RE), where ΔTᵢ is a measured temperature increase at a predetermined time after the laser diagnostic pulse, and where K is an empirically determined calibration constant determined empirically on the basis of the data set.

In one embodiment the controller regulates the source to provide the photothermal pulse to the skin at or below the individual maximum safe radiant exposure (IMSRE) as determined by approximating the statistical regression by an inverse proportionality relationship between individual maximum safe radiant exposures (IMSRE) and a temperature increase ΔT in targeted tissue in the subject induced by a sub-therapeutic laser pulse comprising the temporal radiant energy exposures (RE).

The controller regulates the source to provide the photothermal pulse to the skin at or below the individual maximum safe radiant exposure (IMSRE) as determined by obtaining the separation of the data set with a limitation of 3% or less of the subjects having unacceptable injury at a temporal radiant energy exposure (RE) below the corresponding individual maximum safe radiant exposure (IMSRE).

The controller regulates the source to provide the photothermal pulse to the skin at or below the individual maximum safe radiant exposure (IMSRE) as determined by applying a statistical regression to an empirical data set generated by employing a plurality of measurements over time starting from when a diagnostic laser pulse is applied to approximately one second thereafter to determine individual maximum safe radiant exposure (IMSRE).

The invention also includes a recordable medium for storing instructions for a computer-controlled source of a photothermal pulse to be applied to the skin of a patient with an individual maximum safe radiant exposure (IMSRE) without any use of a biological model comprising instructions for controlling the source to provide a radiant exposure of the skin to the photothermal pulse at or below the individual maximum safe radiant exposure (IMSRE) as determined by using a statistical regression to an empirical data set of individual maximum safe radiant exposures (IMSRE) and temporal radiant energy exposures (RE) applied to a sample population of patients by obtaining a separation of the data set into an acceptable injury grouping and an unacceptable injury grouping with a predetermined limitation of the proportion of subjects having unacceptable injury at a temporal radiant energy exposure (RE) below the corresponding individual maximum safe radiant exposure (IMSRE), from which separation of the data set the individual maximum safe radiant exposure (IMSRE) for a corresponding temporal radiant energy exposure (RE) to the patient has been determined.

The instructions for controlling the source comprise instructions which control the source at or below the individual maximum safe radiant exposure (IMSRE) so as to obtain the separation of the data set with a limitation of 3% or less of the subjects having unacceptable injury at a temporal radiant energy exposure (RE) below the corresponding individual maximum safe radiant exposure (IMSRE).

What is disclosed below is an apparatus and method or methods to process a pulsed photo-thermal radiometric signal into a predicted individual maximum safe radiant exposure value. More specifically, what is disclosed is the calibration itself, including the data set and the K value.

The disclosure recognizes the following causal chain: a higher pigmentation→more laser light absorption→more heat→lower individual maximum safe radiant exposure; and then implements a method on the following principle: a sub-therapeutic laser pulse (low laser energy) induces a small temperature increase which is measured with an Infra-red detector. This provides a measure for the individual's pigmentation and thus for the individual maximum safe radiant exposure.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a symbolic diagram of the prior art problem of how to relate individual pulsed photothermal radiometric (PPTR) measurements to individual maximum safe radiant exposure (IMSRE).

FIG. 2 is a symbolic diagram illustrating the conventional prior art approach of using biological models to solve the problem of FIG. 1.

FIG. 3 is a symbolic diagram of how the illustrated embodiment of the invention relates individual pulsed photothermal radiometric (PPTR) measurements to individual maximum safe radiant exposure (IMSRE).

FIG. 4 shows in an upper graph each of the 304 data points plotted on axes representing the individual maximum safe radiant exposure (IMSRE) and the radiant exposures (RE) used for the test spots. The lower graph of FIG. 4 illustrates the histographic distribution of the data points in the upper graph into four categories of acceptable or unacceptable injury.

FIG. 5 is a graph of the change in temperature of skin as a function of time for two different thicknesses of epidermins.

FIG. 6 shows results of the simulation/feasibility exercise where calculated IMSREs are compared against predicted IMSREs.

FIG. 7 is a pair of graphs in which the upper graph show the feasibility of using partial linear regression to obtain a separation of the data set of points and in which the lower graph shows the corresponding distribution into the four injury categories of FIG. 4.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

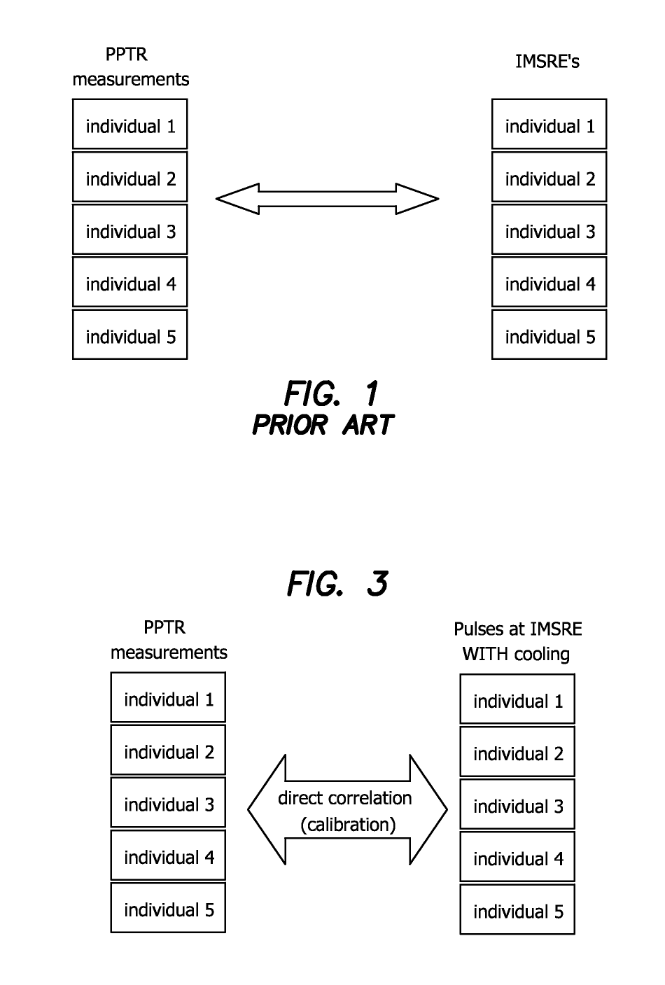

The illustrated embodiment of the invention addresses the question of FIG. 1 of how can an individual pulsed photothermal radiometric measurement be used to determine individual maximum safe radiant exposure. The illustrated embodiments contemplate irradiation of skin by a laser pulse in combination with use of a spurt of cryogenic cooling in a heating/cooling protocol, but it must be expressly understood that the details of the protocol can be widely varied in any given application and in fact the cooling step may be omitted. The irradiation need not be pulsed or from a laser and the cooling need not be cryogenic or even practiced. The concepts of the invention are adaptable to an arbitrary heating and/or cooling methodology of any type of tissue.

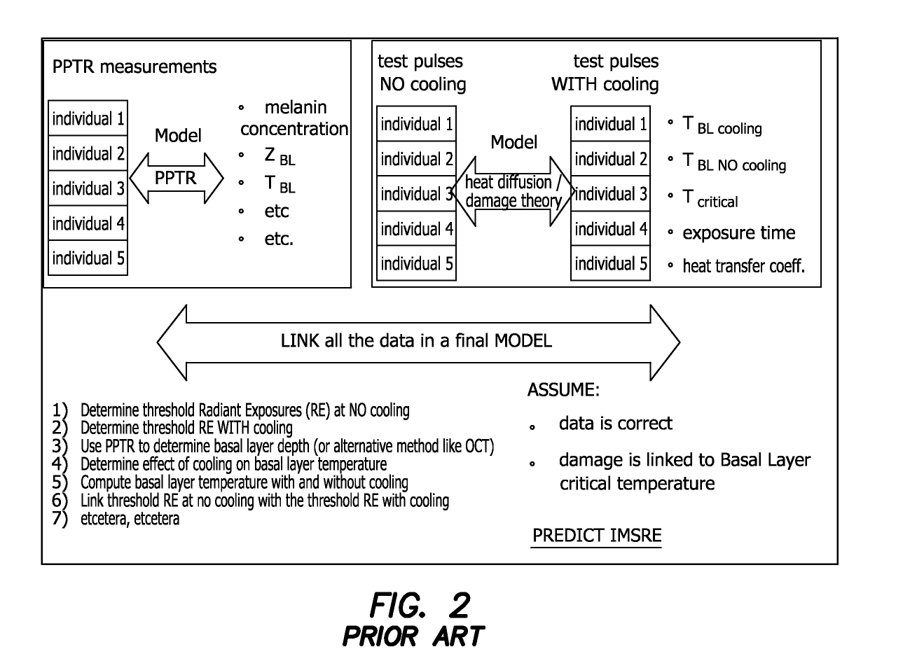

The illustrated method of the invention avoids all the modeling steps of the approach of FIG. 2 and does not require assumption of any values. Instead, it requires calibration with an experimentally determined data set as symbolized by the diagram of FIG. 3.

Two simple embodiments of the method to calibrate illustrate the invention, which are identified below as method #1, and method #2.

Analysis Method #1

The embodiment of method #1 begins with the premise:

$${IMSRE}_{i} = K \frac{{RE}_{D}}{Δ {T}_{i}} , \qquad ( 1 )$$

where RE_{D} is the radiant exposure of the diagnostic laser pulse, ΔTᵢ is the measured temperature increase at 20 ms after the laser pulse and K is a calibration constant (units ° C.). In the illustrated embodiment K was determined empirically on the basis of the data set for 13 volunteers. K is assumed to be a universal constant valid for all skin types involved in the calibration data set and for the laser used, which in this embodiment was a 755 nm laser with 3 ms pulse duration, 50 ms pre cooling spurt duration, and a 30 ms subsequent delay before irradiation. It is to be expressly understood that the wavelength, irradiation period, cooling period and delay interval may be varied among other parameters of the calibration sample population with possible dependency of K thereon. It is expressly to be understood that the illustrated data set in this disclosure is exemplary only and that in any given application that the sample population will be much larger and randomly or representatively selected from the selected target population in order to obtain a valid IMSRE that will be optimally suited for the population to which it is to be applied.

Equation 1 expresses the premise that the individual maximum safe radiant exposure is higher when the temperature increase, which is induced by a sub-therapeutic laser pulse, is lower i.e. IMSRE and temperature are inversely proportional. We chose the ΔT at 20 ms because at this time the contribution of remaining hair follicles on the infra-red signal is negligible while the contribution of the epidermal layer, where the melanin is located, is still dominant over contributions from the deeper chromophores.

On each of the volunteers, test spots were applied with varying radiant exposure (RE), but was intended to be above and beyond the individual maximum safe radiant exposure which we defined as causing visible injury lasting at least 24 hrs.

To determine the K value, we categorize the data points in four categories. Categories 1 and 2 are observed acceptable injuries and categories 3 and 4 are observed unacceptable injuries. Again it must be understood that the definition of “acceptable” and “unacceptable” injury may be modified from that illustrated here without departing from the scope and spirit of the invention. Categories 2 and 4 are at radiant energies in excess of the individual maximum safe radiant exposure and categories 1 and 3 are at radiant energies less than the individual maximum safe radiant exposure. Using this categorization we can determine the optimal K value by minimizing the number of data points in category 2 (acceptable injury, above IMSRE) while the number of data points in category 3 (unacceptable injury, below IMSRE) does not exceed 3% of the total data points. It is also to be understood that the categories definitions can be modified without departing from the spirit and scope of the invention, for example the 3% limitation can be raised or lowered according to desired medical safety limits.

TABLE 1
Categorization of prediction results.

| Category | Injury level | over/under treated |
|---|---|---|
| 1 | acceptable injury | RE_used < individual maximum |
| | | safe radiant exposure |
| 2 | acceptable injury | RE_used > individual maximum |
| | | safe radiant exposure |
| 3 | unacceptable | RE_used < individual maximum |
| | injury found | safe radiant exposure |
| 4 | unacceptable | RE_used > individual maximum |
| | injury found | safe radiant exposure |

The value K is determined such that the number of points at which damage occurs at a radiant exposure (RE) lower than the predicted individual maximum safe radiant exposure (IMSRE) is not more than 3% of the total test spots in the data set, while the individual maximum safe radiant exposure (IMSRE) is maximized at the same time. It followed that for the current data set (in which we used two laser spot sizes: 8 mm and 12 mm) K values of 35 and 27 provided the best prediction. Accuracy of these values can be increased with an expanded calibration data set.

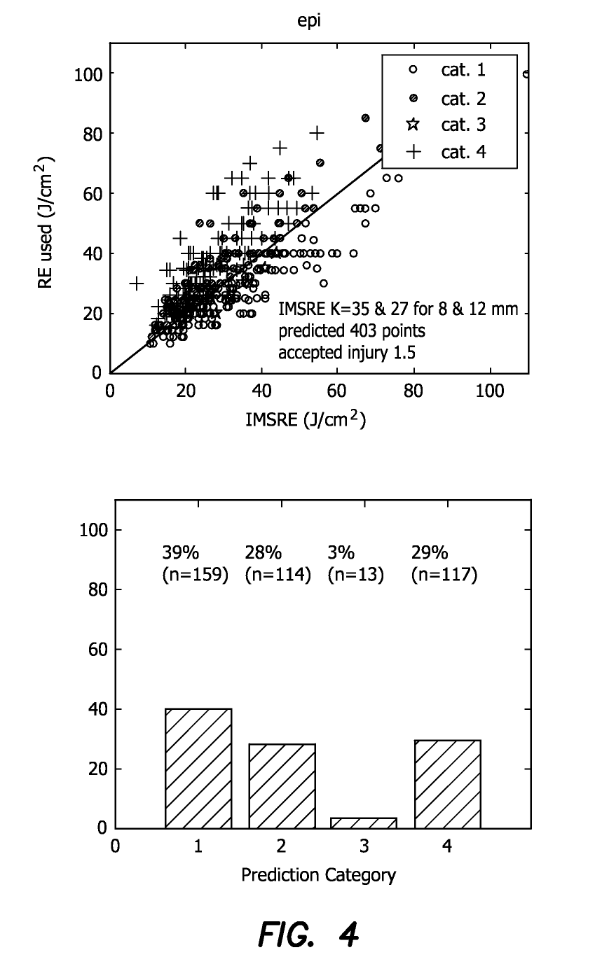

FIG. 4 shows in the upper graph each of the 304 data points plotted on axes representing the individual maximum safe radiant exposure (IMSRE) and the RE used for the test spots. The data points in category 2 and 3 are incorrectly predicted with this method, but in general there is a good separation of points. The lower graph in FIG. 4 shows the fraction of the data points in each of the prediction categories. The majority of the points are correctly predicted.

Analysis Method #2

Whereas method #1 only uses one data point from the pulsed photothermal radiometric signal: ΔT (t=20 ms), the method based on partial least squares regression (PLS) uses the entire pulsed photo-thermal radiometric signal starting from the moment at which the diagnostic laser pulse is applied to about one second later. In other words a time profile or temporal signature of the photo-induced heat production in the skin to the laser pulse/cooling protocol is the measured and characterizing subset of points of the data set.

Method #2, however, is much less intuitive and strongly depends on a mathematical/statistical analysis method, known as partial least squares regression (PLS). Basically, it can quantify the relationship between two known data sets, assuming that there is some linear relationship between these data sets, and then use the resulting data to quantify an unknown value from a known related value.

In our case, the two data sets are the individual maximum safe radiant exposure IMSRE and the pulsed photo-thermal radiometric signals as schematically depicted in FIG. 1. We are interested in determining individual maximum safe radiant exposure from the pulsed photo-thermal radiometric (PPTR) signal.

PLS assumes some degree of linearity between the datasets. The PLS calibration is able to improve the IMSRE prediction based on a PPTR signal, by using information regarding the epidermal thickness, embedded in the PPTR signal. This allows the calibration to account for both pigmentation surface density and pigmentation volumetric density. This condition is satisfied if we use the reciprocal of the pulsed photo-thermal radiometric (PPTR) signal:

Consider first a physical description of the PLS methodology. The pigmentation of skin is a simplification of what is relevant in the prediction of IMSRE because it may refer to the pigmentation surface density (the total amount of melanin per unit skin surface, including the underlying epidermis of that surface), or to the pigmentation volumetric density (melanin per unit volume within the epidermis). This difference would be irrelevant if all human epidermis were the same thickness. However, epidermal thickness can vary from approximately 50 micrometers to approximately 200 micrometers in different locations. Assume two skin areas with equal pigmentation surface density but with epidermal thicknesses of 50 and 100 micrometers, respectively, the melanin per unit volume would in the latter epidermis would be only half that in the former epidermis. In other words, the concentration of melanin is different by a factor of two. It follows that the absorption of laser light by melanin and subsequent heat production per unit volume is also different by a factor of two. If the heat production per unit volume is different by a factor of two, it follows that peak temperatures are also different. Thermal heat diffusion, during the laser pulse, will cause the peak temperatures to differ by a factor less than two, although a difference in (peak) temperature will still be affected. The above example is to illustrate that the total melanin content per unit skin surface may not be as relevant for the prediction of the IMSRE as the melanin content per unit volume.

Existing apparatus quantify individual pigmentation as a single number, the so called “melanin index” (e.g. the Mexameter by Courage-Khazaka Electronic, Cologne, Germany). It is our understanding that these devices provide a quantification for the pigmentation surface density and disregard the effect of epidermal thickness. Our data as well as our understanding of the thermally induced skin injury suggests that a more precise prediction of the IMSRE should involve a quantification of the epidermal thickness as well. A PPTR measurement contains information regarding the thickness of the epidermis, and can thus provide a measure for not only the pigmentation surface density but the volumetric density as well.

If a PPTR measurement is performed on a relatively thick epidermis, the temperature signal will drop less fast than if it were on a relatively thin epidermis due to the larger thermal relaxation time for the thicker epidermis.

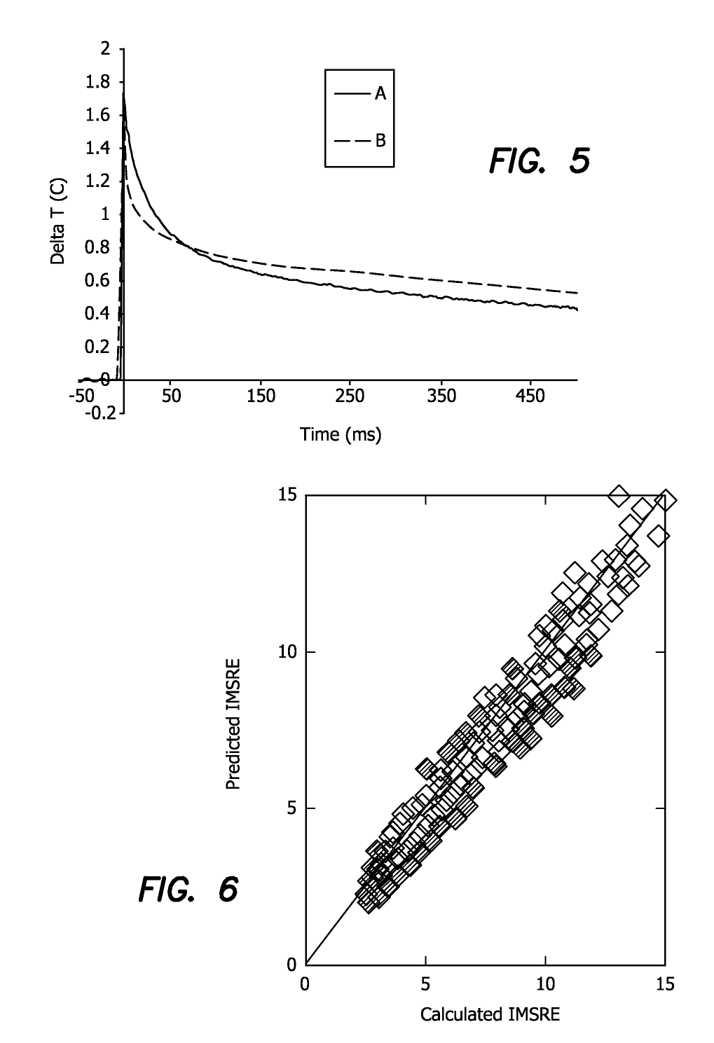

Examples of measured PPTR signals with probably different epidermal thicknesses are shown in the graph of FIG. 5. The PPTR signal A shows a relatively rapid decline with time, indicating a relatively thin epidermis with a small thermal relaxation time. PPTR signal B shows a slower decline with time, indicating a thicker epidermis with a larger thermal relaxation time. The larger temperature increase of the PPTR signal B for times >50 ms indicates a

A prior art model approach as illustrated in FIG. 2 would attempt to quantify the epidermal thickness and then apply a damage model to calculate the expected injuries for these different epidermal geometries.

In contrast, a calibration with PLS uses these signals and lets the mathematical, statistical algorithm determine how the shapes of these PPTR signals correlate with the IMSRE. A more statistically oriented explanation of the PLS methodology is as follows. The analysis method #1 (using a single k value and the PPTR signal at 20 ms) uses only one point of the PPTR signal, and basically uses linear regression to calibrate the IMSRE prediction. We could now expand this method to also use the PPTR signal at 30 ms and improve the prediction by performing multiple linear regression, using the 20 ms and 30 ms as data points. Expanding this even further would use each time point in the PPTR signal and use multiple linear regression to find the best constants for each of these time points. PLS is doing essentially exactly that. An important difference with multiple linear regression, however, is that PLS uses a factor based approach to perform a quantitative calibration. This or similar techniques are also often referred to as principal component regression.

If each of the individually measured pulsed photothermal radiometric signals Δ(t) are written as vectors T_, their reciprocal can be written as

T=1/T_. (2)

The length of the vector T is for example 1000 if we sampled the pulsed photo-thermal radiometric signal at 1000 Hz and acquired the signal for one second.

In terms of linear algebra we can now write:

IMSRE_{I}=KT_{I} (3),

where K is a vector of the same length as T such that the matrix product KTᵢ equals IMSREᵢ

The problem now is to find the vector K which is needed to use equation 3 in order to predict the individual maximum safe radiant exposure IMSRE with a measured pulsed photothermal radiometric signal. This is briefly described below

PLS provides K in a calibration step. We first identify all test spots in which we just reached the damage threshold. We use the RE values that caused this threshold as the individual maximum safe radiant exposure IMSRE, forming a vector I. The associated reciprocal pulsed photothermal radiometric signals T (defined in equation 2) form a matrix T.

The calibration step in PLS, which is a conventional well known algorithm, uses I and T to produce K.

In the prediction step PLS essentially uses equation 3 to determine the unknown IMSREᵢ for any measured signal Tᵢ.

We tested PLS for our application using simulated pulsed photothermal radiometric signals to investigate the feasibility of PLS. Determining feasibility was necessary because using PLS for temporal data instead of spectral data is highly unusual and the results could not be assumed to be correct. We tested the PLS algorithm for our purpose because this technique is typically used to extract concentrations of a chemical from a measured (absorption or reflectance) spectrum. The application of PLS for temporal signals has not been previously done. Simulation confirmed that PLS was a feasible approach and later validation with experimental data as well confirmed it.

Although PLS is specifically used in the illustrated embodiment, it must be understood that any statistical regression technique may be applied that gives satisfactory results. We have thus far only used PLS, but other statistical regression methods may work just as well or better. The relevant point is that the approach of the invention is model free. No assumptions need to be made, nor additional modeling or reconstructions are necessary. This is the underlying mechanism for the robustness of the method.

FIG. 6 shows results of the simulation/feasibility exercise where calculated IMSREs are compared against predicted IMSREs. Simulated use of PLS to predict individual maximum safe radiant exposure from a pulsed photothermal radiometric signal is shown in FIG. 6. Pulsed photothermal radiometric signals were simulated for a variety of skin pigmentations and epidermal thicknesses. For these same skin geometries, laser treatment was modeled with and without cooling. The individual maximum safe radiant exposure was calculated by assuming a critical threshold temperature for damage at the basal layer. PLS was used to predict the individual maximum safe radiant exposures (vertical axis of FIG. 6) from the pulsed photothermal radiometric signals and was then compared with those calculated. The results indicate the feasibility of PLS for this application.

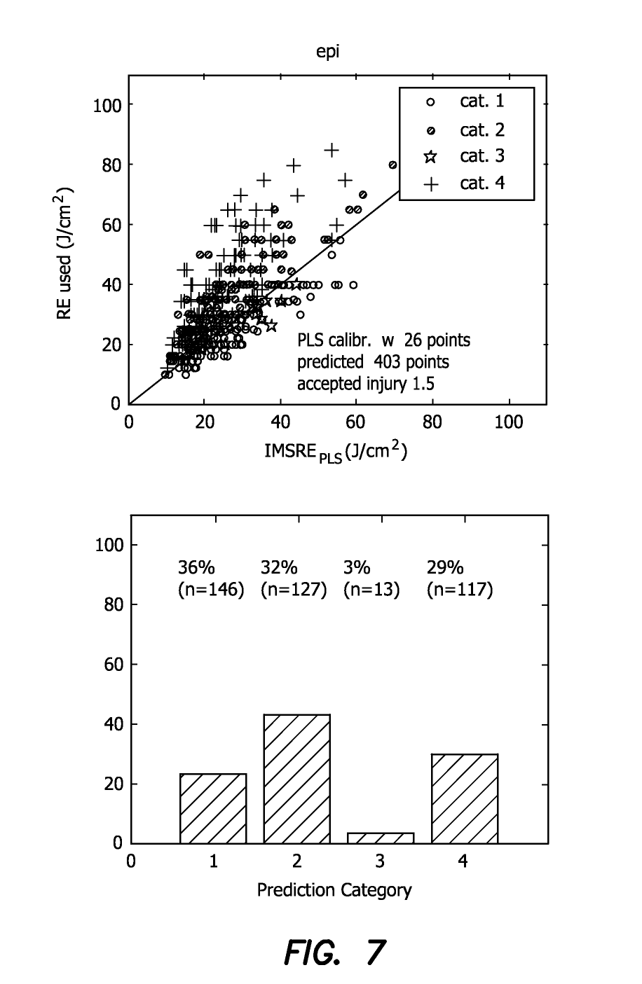

We have used the experimentally acquired data to perform our first PLS calibration and then used the result to apply on the entire set of experimentally obtained data to verify the feasibility of PLS as shown in FIG. 7. The same data points of all 13 volunteers (403 data points in total) are plotted on the same axes as in FIG. 4. The PLS predicted individual maximum safe radiant exposure (method #2) are clearly different than those with the simpler method #1. Note that the prediction seems to be inaccurate for higher individual maximum safe radiant exposure values. However, we are confident this is only due to the fact that the calibration data set is relatively underrepresented for this region. What is important to notice in the upper graph of FIG. 7 is that the points are much better separated than in the upper graph in FIG. 4. This is what is important for an accurate individual maximum safe radiant exposure prediction. We are confident that with an extended calibration data set, the category 2 points would be drastically reduced while the category 3 points would be the same or reduced as well.

Even without an extended calibration set, a pragmatic user of FIG. 7 would simply draw an empirical line (other than the straight line) between the data points which would already improve the prediction quality.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. An improvement in a method for making pulsed photothermal radiometric measurements to determine individual maximum safe radiant exposure (IMSRE) of biological subjects corresponding to radiant energy exposure (RE) without any use of a biological model comprising:
generating an empirical data set of individual maximum safe radiant exposures (IMSRE) and temporal radiant energy exposures (RE) applied to a sample population of the subjects;
applying a statistical regression to the data set to determine a individual maximum safe radiant exposure (IMSRE) corresponding to each temporal radiant energy exposure (RE) by obtaining a separation of the data set into an acceptable injury grouping and an unacceptable injury grouping with a predetermined limitation of the proportion of subjects having unacceptable injury at a temporal radiant energy exposure (RE) below the corresponding individual maximum safe radiant exposure (IMSRE); and
utilizing the separation of the data set to predict an individual maximum safe radiant exposure (IMSRE) for a corresponding temporal radiant energy exposure (RE) to a biological subject not included in the sample population.

2. The improvement of claim 1 where applying a statistical regression to an empirical data set of individual maximum safe radiant exposures (IMSRE) and temporal radiant energy exposures (RE) to determine a individual maximum safe radiant exposure (IMSRE) corresponding to each temporal radiant energy exposure (RE) comprises applying partial least squares (PLS) regression to quantify a relationship between the individual maximum safe radiant exposures (IMSRE) and temporal radiant energy exposures (RE) in the data set.

3. The improvement of claim 1 where applying a statistical regression to an empirical data set of individual maximum safe radiant exposures (IMSRE) and temporal radiant energy exposures (RE) to determine a individual maximum safe radiant exposure (IMSRE) corresponding to each temporal radiant energy exposure (RE) comprises approximating the statistical regression by the relation:
$${IMSRE}_{i} = K \frac{{RE}_{D}}{Δ {T}_{i}} , \qquad ( 1 )$$
where RE_{D} is a radiant exposure of a diagnostic laser pulse, which comprises the temporal radiant energy exposure (RE), where ΔTᵢ is a measured temperature increase at a predetermined time after the laser diagnostic pulse, and where K is an empirically determined calibration constant determined empirically on the basis of the data set.

4. The improvement of claim 3 where radiant exposure is made to skin of the subject having hair follicles, an epidermal layer and deeper chromophores, and where the predetermined time after the laser diagnostic pulse comprises a time period at which contribution to heat absorption in the skin from the hair follicles is negligible while contribution to heat absorption in the skin from the melanin bearing epidermal layer is dominant over contribution to heat absorption in the skin from deeper chromophores.

5. The improvement of claim 3 where the predetermined time after the single laser diagnostic pulse comprises a measurement at approximately 20 ms.

6. The improvement of claim 5 where the predetermined time after the single laser diagnostic pulse comprises a single measurement at approximately 20 ms.

7. The improvement of claim 1 where applying a statistical regression to an empirical data set of individual maximum safe radiant exposures (IMSRE) and temporal radiant energy exposures (RE) to determine a individual maximum safe radiant exposure (IMSRE) corresponding to each temporal radiant energy exposure (RE) comprises approximating the statistical regression by an inverse proportionality relationship between individual maximum safe radiant exposures (IMSRE) and a temperature increase ΔT in targeted tissue in the subject induced by a sub-therapeutic laser pulse comprising the temporal radiant energy exposures (RE).

8. The improvement of claim 1 where applying a statistical regression to an empirical data set of individual maximum safe radiant exposures (IMSRE) and temporal radiant energy exposures (RE) to obtain a separation of the data set into an acceptable injury grouping and an unacceptable injury grouping with a predetermined limitation of the proportion of subjects having unacceptable injury at a temporal radiant energy exposure (RE) below the corresponding individual maximum safe radiant exposure (IMSRE) comprises obtaining the separation of the data set with a limitation of 3% or less of the subjects having unacceptable injury at a temporal radiant energy exposure (RE) below the corresponding individual maximum safe radiant exposure (IMSRE).

9. The improvement of claim 1 where applying a statistical regression to an empirical data set of individual maximum safe radiant exposures (IMSRE) and temporal radiant energy exposures (RE) applied to a sample population of the subjects to determine a individual maximum safe radiant exposure (IMSRE) corresponding to each temporal radiant energy exposure (RE) comprises applying a statistical regression to an empirical data set generated by employing a plurality of measurements over time starting from when a diagnostic laser pulse is applied to approximately one second thereafter to determine individual maximum safe radiant exposure (IMSRE).

10. The improvement of claim 9 where applying a statistical regression comprises using partial least squares regression (PLS) to determine an individual maximum safe radiant exposure vector (IMSREᵢ) whose components are individual maximum safe radiant exposure values from the data set in which a predetermined damage threshold is just reached, where RE values that caused the predetermined damage threshold are used as the individual maximum safe radiant exposure values, and where Tᵢ is a vector whose components are reciprocal pulsed photo-thermal radiometric signals Tᵢ corresponding to the individual maximum safe radiant exposure values in IMSREᵢ, where K is a vector having the same length as Tᵢ and is determined using PLS from IMSRE_{I}=K×T_{I}, where IMSREᵢ is the matrix product K×Tᵢ.

11. A method for applying a photothermal pulse to the skin of a patient with an individual maximum safe radiant exposure (IMSRE) without any use of a biological model comprising applying the photothermal pulse to the skin with a radiant exposure at or below the individual maximum safe radiant exposure (IMSRE) as determined by using a statistical regression to an empirical data set of individual maximum safe radiant exposures (IMSRE) and temporal radiant energy exposures (RE) applied to a sample population of patients by obtaining a separation of the data set into an acceptable injury grouping and an unacceptable injury grouping with a predetermined limitation, of the proportion of subjects having unacceptable injury at a temporal radiant energy exposure (RE) below the corresponding individual maximum safe radiant exposure (IMSRE), from which separation of the data set the individual maximum safe radiant exposure (IMSRE) for a corresponding temporal radiant energy exposure (RE) to the patient has been determined.

12. An apparatus comprising:
a source of a photothermal pulse to be applied to the skin of a patient with an individual maximum safe radiant exposure (IMSRE) without any use of a biological model;
a controller coupled to the source where the radiant exposure provided by the photothermal pulse to the skin from the source as regulated by the controller is maintained at or below the individual maximum safe radiant exposure (IMSRE) as determined by using a statistical regression to an empirical data set of individual maximum safe radiant exposures (IMSRE) and temporal radiant energy exposures (RE) applied to a sample population of patients by obtaining a separation of the data set into an acceptable injury grouping and an unacceptable injury grouping with a predetermined limitation of the proportion of subjects having unacceptable injury at a temporal radiant energy exposure (RE) below the corresponding individual maximum safe radiant exposure (IMSRE), from which separation of the data set the individual maximum safe radiant exposure (IMSRE) for a corresponding temporal radiant energy exposure (RE) to the patient has been determined.

13. The apparatus of claim 12 where the controller regulates the source to provide the photothermal pulse to the skin at or below the individual maximum safe radiant exposure (IMSRE) as determined by applying partial least squares (PLS) regression to quantify a relationship between the individual maximum safe radiant exposures (IMSRE) and temporal radiant energy exposures (RE) in the data set.

14. The apparatus of claim 12 where the controller regulates the source to provide the photothermal pulse to the skin at or below the individual maximum safe radiant exposure (IMSRE) as determined by approximating the statistical regression by the relation:
$${IMSRE}_{i} = K \frac{{RE}_{D}}{Δ {T}_{i}} , \qquad ( 1 )$$
where RE_{D} is a radiant exposure of a diagnostic laser pulse, which comprises the temporal radiant energy exposure (RE), where ΔTᵢ is a measured temperature increase at a predetermined time after the laser diagnostic pulse, and where K is an empirically determined calibration constant determined empirically on the basis of the data set.

15. The apparatus of claim 14 where radiant exposure is made to skin of the subject having hair follicles, an epidermal layer and deeper chromophores, and where the predetermined time after the laser diagnostic pulse comprises a time period at which contribution to heat absorption in the skin from the hair follicles is negligible while contribution to heat absorption in the skin from the melanin bearing epidermal layer is dominant over contribution to heat absorption in the skin from deeper chromophores.

16. The apparatus of claim 12 where the controller regulates the source to provide the photothermal pulse to the skin at or below the individual maximum safe radiant exposure (IMSRE) as determined by approximating the statistical regression by an inverse proportionality relationship between individual maximum safe radiant exposures (IMSRE) and a temperature increase ΔT in targeted tissue in the subject induced by a sub-therapeutic laser pulse comprising the temporal radiant energy exposures (RE).

17. The apparatus of claim 12 where the controller regulates the source to provide the photothermal pulse to the skin at or below the individual maximum safe radiant exposure (IMSRE) as determined by obtaining the separation of the data set with a limitation of 3% or less of the subjects having unacceptable injury at a temporal radiant energy exposure (RE) below the corresponding individual maximum safe radiant exposure (IMSRE).

18. The apparatus of claim 12 where the controller regulates the source to provide the photothermal pulse to the skin at or below the individual maximum safe radiant exposure (IMSRE) as determined by applying a statistical regression to an empirical data set generated by employing a plurality of measurements over time starting from when a diagnostic laser pulse is applied to approximately one second thereafter to determine individual maximum safe radiant exposure (IMSRE).

19. A recordable medium for storing instructions for a computer-controlled source of a photothermal pulse to be applied to the skin of a patient with an individual maximum safe radiant exposure (IMSRE) without any use of a biological model comprising instructions for controlling the source to provide a radiant exposure of the skin to the photothermal pulse at or below the individual maximum safe radiant exposure (IMSRE) as determined by using a statistical regression to an empirical data set of individual maximum safe radiant exposures (IMSRE) and temporal radiant energy exposures (RE) applied to a sample population of patients by obtaining a separation of the data set into an acceptable injury grouping and an unacceptable injury grouping with a predetermined limitation of the proportion of subjects having unacceptable injury at a temporal radiant energy exposure (RE) below the corresponding individual maximum safe radiant exposure (IMSRE), from which separation of the data set the individual maximum safe radiant exposure (IMSRE) for a corresponding temporal radiant energy exposure (RE) to the patient has been determined.

20. The recordable medium of claim 19 where the instructions for controlling the source comprise instructions which control the source at or below the individual maximum safe radiant exposure (IMSRE) so as to obtain the separation of the data set with a limitation of 3% or less of the subjects having unacceptable injury at a temporal radiant energy exposure (RE) below the corresponding individual maximum safe radiant exposure (IMSRE).
